(19) United States
(12) Patent Application Publication
(10) Pub. No.: US 2023/0251267 A1
(43) Pub. Date: Aug. 10, 2023

(54) OPTIMIZED DETECTION OF BLOOD-BORNE MICROBES

(71) Applicant: Peter Vincent Pickens, Wayne, PA (US)

(72) Inventor: Peter Vincent Pickens, Wayne, PA (US)

(21) Appl. No.: 18/299,967

(22) Filed: Apr. 13, 2023

Related U.S. Application Data
(63) Continuation-in-part of application No. PCT/US21/54934, filed on Oct. 14, 2021.
(60) Provisional application No. 63/091,471, filed on Oct. 14, 2020.

(51) Int. Cl. G01N 33/68; G01N 33/569; C12Q 1/04

(52) U.S. Cl. CPC G01N 33/6848; G01N 33/56911; C12Q 1/04

(57) ABSTRACT

Traditional methods of detecting blood-borne microbial species in patients suspected of sepsis can be relatively slow, lack sensitivity, and require large volumes of blood. The present invention relates to methods of detecting microbial species in platelet rich plasma, which can be done more rapidly, with greater sensitivity, and with smaller volumes of blood to ensure more prompt and reliable diagnosis and treatment. The present invention also relates to improved methods of antibiotic treatment for patients diagnosed with a microbial infection and improved methods of excluding the diagnosis of viral infection exhibiting symptoms similar to sepsis.

### CROSS-REFERENCE TO RELATED APPLICATIONS

This application is a continuation-in-part of International Patent Application No. PCT/US2021/054934, filed Oct. 14, 2021, which claims priority to U.S. Provisional Patent Application No. 63/091,471 filed on Oct. 14, 2020, the contents of each of which are incorporated by reference herein in their entirety.

### BACKGROUND OF THE INVENTION

Blood stream infections, which, if severe enough, can lead to sepsis, are among the top 7 causes of death in the USA and Europe. 1.2 million cases of blood borne bacterial infections occur in Europe annually, accounting for 157,000 deaths (Lamy, B., et al. 2016. Frontiers in Microbiology, 7(697): 1-13). Sepsis is a life-threatening condition and is defined clinically as a strong suspicion for severe microbiologic infection, with symptoms including increased heart rate, fever, and elevated or decreased blood pressure. The term septic shock indicates the development of end-organ failure, such as renal, cardiac, hepatic or respiratory failure (Singer, M., et al. 2016. JAMA, 315(8): 801-810; Alam, N., et al. 2018. The Lancet Respiratory Medicine, 6(1): 40-50). Bacterial sepsis, and its closely related clinical entity septic shock, continue to represent a severe global health care burden. Mortality associated with sepsis or septic shock remains disturbingly high, with estimates between 15% and 40% even in the face of aggressive management (Assinger, A., et al. 2019. Frontiers in Immunology, 10(1687): 1-19; Alam, N., et al. 2018. The Lancet Respiratory Medicine, 6(1): 40-50; Brooks, D., et al. 2016. American Journal of Infection Control, 44(11): 1291-1295). Despite numerous clinical trials employing a variety of potentially promising agents, the mortality associated with sepsis and septic shock has not improved in recent decades. Investigators have noted a troubling increase in the incidence of sepsis and septic shock worldwide, due to the aging population, the increasing use of immunosuppressive drugs in the treatment of cancer and autoimmune diseases, and improved recognition of the syndrome (Alam, N., et al. 2018. The Lancet Respiratory Medicine, 6(1): 40-50). Treatment has varied little in recent decades, and includes prompt initiation of appropriate antibiotics, fluid resuscitation, blood pressure support with inotropic agents that improve blood pressure (BP) and organ perfusion, and ventilatory support to provide sufficient oxygenation of blood. Of all these interventions, antibiotic treatment of bacterial infections is by far the most important.

In order to provide appropriate and specific antibiotic treatment to optimize patient management, cultures of blood are performed in all patients in the hopes of providing precise microbiologic information. Identification of the precise bacterium at hand can then inform the selection of appropriate and specific antibiotic treatment, thus maximizing the patient's chance for survival (Kumar, A., et al. 2009. Chest, 136(5): 1237-1248; Phua, J., et al. 2013. Critical Care, 17(202): 1-12). Unfortunately, blood cultures utilizing whole blood are woefully inadequate in providing key microbiologic information. In fact, the sensitivity of blood cultures performed in the traditional manner in patients with sepsis ranges from just 32% up to 71%, with most studies reporting a sensitivity of under 50% (Alam, N., et al. 2018. The Lancet Respiratory Medicine, 6(1): 40-50; Kumar, A., et al. 2009. Chest, 136(5): 1237-1248; Bernard, G. R., et al. 2001. Critical Care Medicine, 29(11): 2051-2059; Phua, J., et al. 2013. Critical Care, 17(202): 1-12; Brooks, D., et al. 2016. American Journal of Infection Control, 44(11): 1291-1295). In the remaining patients, clinicians typically provide a “shotgun” antibiotic approach, administering two to as many as four different antibiotics depending upon clinical factors so as to cover a variety of bacteria that can result in the patient's specific clinical presentation. Kumar, et al. have demonstrated that inappropriate selection of antibiotics in patients with sepsis results in a fivefold increase in mortality (Kumar, A., et al. 2009. Chest, 136(5): 1237-1248). Negative blood cultures, despite overt clinical sepsis, are particularly problematic in women, and in all patients with pneumonia (Phua, J., et al. 2013. Critical Care, 17(202): 1-12). Studies recently published in the medical literature clearly indicate that the microbiologic information provided by a positive blood culture translates into the informed selection of appropriate antibiotics, thereby reducing patient mortality and morbidity, and reducing health care costs and hospital length of stay (Kumar, A., et al. 2009. Chest, 136(5): 1237-1248). Thus, there is a desperate need to improve the sensitivity of blood cultures in patients with sepsis and septic shock.

Bacterial sepsis and septic shock share many clinical similarities with severe acute respiratory syndrome coronavirus 2 (SARS-CoV-2) infection, as has been learned during the global coronavirus 2019 (COVID-19) pandemic. Specifically, patients with severe COVID-19 caused by SARS-CoV-2 infection similarly display high fever, increased heart rate, elevated or low blood pressure, and cardiovascular collapse with end organ failure (Berlin, D. A., et al. 2020. New England Journal of Medicine: 1-10). To differentiate bacterial sepsis from severe COVID-19, blood cultures are performed on all patients displaying these symptoms at presentation. The detection of circulating bacteria in the bloodstream of such patients would therefore inform treating clinicians of the bacterial origin of the patient's presentation, and thereby help exclude a diagnosis of active SARS-CoV-2 infection. Thus, improvement in blood culture methodology would improve clinician's ability to promptly exclude COVID-19.

In traditional blood culture techniques, 20 to 40 mL of whole blood is drawn from a peripheral vein by venipuncture and injected into a blood culture bottle. The medium within these bottles promotes and sustains bacterial growth. The culture bottle is then incubated for 5 days at between 33.5 and 36.5 degrees Centigrade. Cultures are reviewed continuously for bacterial growth. If bacterial growth is detected, fluid from the culture bottle is removed and plated on a substrate (e.g. a petri dish) to further characterize the bacterium, and define its antibiotic sensitivity (Lamy, B., et al. 2016. Frontiers in Microbiology, 7(697): 1-13; Dawson, S. 2014. Journal of Hospital Infection, 87(1), 1-10). This technique of using whole blood to perform blood cultures as outlined above has not changed appreciably in many decades. Given the frequent failure of traditional whole blood culture techniques to detect bacteria circulating in the blood (estimates of positive detection ranging from 32% to 71% of cases), new and more sensitive techniques are needed to identify the presence or absence of microbes in patients presenting with symptoms related to sepsis and septic shock (Alam, N., et al. 2018. The Lancet Respiratory Medicine, 6(1): 40-50; Kumar, A., et al. 2009. Chest, 136(5): 1237-1248; Bernard, G. R., et al. 2001. Critical Care Medicine, 29(11): 2051-2059; Phua, J., et al. 2013. Critical Care, 17(202): 1-12; Brooks, D., et al. 2016. American Journal of Infection Control, 44(11): 1291-1295).

Thus, there is a need in the art for improved methods of detecting microbes present in the blood of suspected septic patients. This invention satisfies this unmet need.

### SUMMARY OF THE INVENTION

In one embodiment, the present invention comprises a method of detecting one or more blood-borne microbes in a subject at risk of microbial infection, comprising the steps of: obtaining platelet rich plasma (PRP) from a sample of whole blood drawn from said subject; incubating the PRP at a temperature greater than room temperature for up to 5 days, wherein said incubating step occurs less than 24 hours after said obtaining step; and detecting the presence of one or more blood-borne microbes in said PRP.

In one embodiment of the method, said one or more blood-borne microbe comprises a pathogenic microbe. In one embodiment, said pathogenic microbe comprises a bacterium. In one embodiment, said subject exhibits symptoms of sepsis. In one embodiment, said subject comprises a human.

In one embodiment, the method of the present invention further comprises contacting said whole blood sample with one or more anticoagulant prior to obtaining PRP. In one embodiment, said anticoagulant comprises one or more selected from the group consisting of: an ethylenediaminetetraacetic acid (EDTA) salt, a heparin salt, a citrate salt, an oxalate salt, and acid citrate dextrose (ACD). In one embodiment, the anticoagulant comprises a heparin salt.

In one embodiment of the method, the volume of said whole blood sample from said subject comprises a volume that is substantially less than is recommended for blood cultures utilizing whole blood. In one embodiment, said sample of whole blood drawn is less than 20 milliliters in volume. In one embodiment, said volume reduces contamination and false positive detection of blood-borne microbes as compared to blood cultures utilizing whole blood.

In one embodiment, the method of the present invention further comprises the step of contacting said whole blood sample with an agent that induces a change in the shape of platelets from biconcave discs to spherical.

In one embodiment of the method, said detecting step comprises mass spectrometric analysis.

In one embodiment of the method, the rate of true positive detection of blood-borne microbes is greater than that of blood cultures utilizing whole blood. In one embodiment, the sensitivity of detection of blood-borne microbes is greater than that of blood cultures utilizing whole blood. In one embodiment, the time to detection of blood-borne microbes is less than that of blood cultures utilizing whole blood.

In one embodiment, the present invention comprises a method of selecting a therapeutic specific to one or more blood-borne microbe in a subject at risk of microbial infection, comprising the steps of: obtaining platelet rich plasma (PRP) from a sample of whole blood drawn from said subject; incubating the PRP at a temperature greater than room temperature for up to 5 days, wherein said incubating step occurs less than 24 hours after said obtaining step; detecting the presence of one or more blood-borne microbes in said PRP; and selecting one or more therapeutic that will specifically inhibit or destroy one or more blood-borne microbe detected in the PRP of said subject. In one embodiment, said therapeutic is an antibiotic.

In one embodiment, the present invention comprises a method of treating a subject with a microbial infection, comprising the steps of: obtaining platelet rich plasma (PRP) from a sample of whole blood drawn from said subject; incubating the PRP at a temperature greater than room temperature for up to 5 days, wherein said incubating step occurs less than 24 hours after said obtaining step; detecting the presence of one or more blood-borne microbes in said PRP; and selecting one or more therapeutic that will specifically inhibit or destroy one or more blood-borne microbe detected in the PRP of said subject; and treating said subject with a therapeutically effective amount of one or more antibiotic specific to one or more blood-borne microbe detected in the PRP of said subject.

In one embodiment of the method, said antibiotic selection results in fewer non-specific antibiotics being used to treat said subject as compared to antibiotic selection based upon blood cultures utilizing whole blood. In one embodiment, said antibiotic selection reduces the risk of drug-reacted morbidities as compared to antibiotic selection based upon blood cultures utilizing whole blood.

In one embodiment of the method, said detection of the presence of one or more microbes excludes the diagnosis of a viral infection that presents with similar symptoms. In one embodiment, said viral infection is severe acute respiratory syndrome coronavirus 2 (SARS-CoV-2) infection.

### DETAILED DESCRIPTION

The present invention generally relates to improved methods for detecting blood-borne microbes in patients. The present invention is based, in part, on the discovery that cultures utilizing platelet rich plasma, separated and isolated from whole blood samples, can more rapidly and more sensitively detect microbial infection than state of the art whole blood cultures, while also requiring smaller volumes of blood. In some embodiments, the patient presents with symptoms of sepsis. In some embodiments, the patient is administered antibiotics specific to the microbe detected to treat sepsis. In some embodiments, the detection of a microbial infection can be used to rule out a viral infection that presents with similar symptoms.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

“Antibiotics”, as used herein, refer to any chemical substance that has the capacity to inhibit the growth of or destroy a microbe. For example, antibiotics may be used to treat an infection of a disease-causing bacterium.

As used herein, “blood” means whole blood or any fraction thereof, for example plasma, platelets, or a concentrated suspension of cells, such as platelet rich plasma, derived from the circulation of a subject. “Whole blood” refers to an unfractionated suspension of blood components, including red blood cells, white blood cells, platelets, and proteins suspended in plasma.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, “detection” refers to both quantitative and qualitative measurement to determine if a species, characteristic, trait, or feature is present or not. Detection may be relative or absolute. “Detecting the presence of” includes determining the amount of something present, as well as determining whether it is present or absent.

“False positive detection” refers to the determination of the presence of a species, characteristic, trait, or feature when it is not, in fact, truly present. “False negative detection” refers to the determination of the absence of a species, characteristic, trait, or feature when it is, in fact, truly present. “True positive detection” refers to the determination of the presence of a species, characteristic, trait, or feature when it is, in fact, truly present. “True negative detection” refers to the determination of the absence of a species, characteristic, trait, or feature when it is, in fact, truly absent.

An “effective amount” as used herein, means an amount which provides a therapeutic or prophylactic benefit. “Instructional material,” as that term is used herein, includes a publication, a recording, a diagram, or any other medium of expression which can be used to communicate the usefulness of the composition or method of the invention. Optionally, or alternately, the instructional material may describe one or more methods of identifying, diagnosing or alleviating the diseases or disorders in a cell or a tissue of a subject. The instructional material of the kit may, for example, be affixed to a container that contains the composition of the invention or be shipped together with a container that contains the composition. Alternatively, the instructional material may be shipped separately from the container with the intention that the recipient uses the instructional material and the composition cooperatively.

“Isolated” means altered or removed from the natural state. For example, a cell or cells naturally present in a living animal is not “isolated,” but the same cell or cells partially or completely separated from the coexisting materials of its natural state is “isolated.” As a further example, platelets and plasma that are separated from red blood cells and white blood cells are considered isolated from the whole blood sample from which they are derived.

As used herein, the term “microbe” refers to a microscropic, unicellular organism, such as bacteria, fungi, or protozoa. “Microbial infection” herein refers to the invasion of a microbe that causes a disease or disorder.“Pathogenic”, as used herein, refers to the ability of a microbe to cause a microbial infection.

The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a human.

As used herein, “platelets” refers to a preparation or sample enriched for platelet cells, microparticles, or a combination thereof. “Platelet rich plasma” refers to a preparation or sample that is enriched for platelet cells but remains suspended in blood plasma. For example, platelet rich plasma can be isolated from whole blood by centrifugation, separating out the denser red and white blood cells.

As used herein, “sample” means any sample of biological material derived from a subject, such as, but not limited to, blood, plasma, mucus, biopsy specimens and fluid, which has been removed from the body of the subject. The sample which is tested according to the method of the present invention may be tested directly or indirectly and may require some form of treatment prior to testing. For example, a blood sample may require one or more separation steps prior to testing. Further, to the extent that the biological sample is not in liquid form, (for example it may be a solid, semi-solid or a dehydrated liquid sample) it may require the addition of a reagent, such as a buffer, to mobilize the sample.

The term “sensitivity”, as used herein, refers to the proportion of positives that are correctly identified. For example, a diagnostic that can more accurately determine true positive detection of a bacterial infection than another diagnostic has greater sensitivity.

“Specific”, as used herein in reference to antibiotics, refers to the ability of an antibiotic to specifically inhibit or destroy a microbe or microbes with particular characteristics. For example, some antibiotics may be specific to gram-negative or gram-positive bacteria. It would therefore be non-ideal to treat a patient infected with a gram-negative bacterium with an antibiotic specific to gram-positive bacteria, or vice versa. A “non-specific” antibiotic is one that does not have the ability to inhibit or destroy a microbe or microbes to which it is directed or broadly inhibits or destroys a wide range of microbes.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease or disorder conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease or disorder and its severity and the age, weight, etc., of the subject to be treated.

To “treat” a disease or disorder as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based, in part, on the growing body of evidence suggesting that platelets play a key role in innate immunity. The present invention is also based, in part, on the long felt but currently unmet need to improve the accuracy and expediency of detecting blood-borne pathogens in patients. The present invention satisfied this unmet need, as it relates the discovery that cultures utilizing platelet rich plasma, isolated from whole blood, can more sensitively and more rapidly detect bacterial infection from smaller volumes than cultures utilizing whole blood, which has been the standard of the art for many decades.

### Sample Preparation

In one embodiment, the present invention comprises an improved method for detecting blood-borne microbes in patients at risk of microbial infection, comprising the steps of: 1) obtaining blood from the patient, 2) isolating platelet rich plasma (PRP) from said patient, 3) incubating said PRP, and 4) detecting the presence of microbes in said PRP.

The process of obtaining blood for cultures to detect bacterial infection, as it relates to the present invention, can be performed using numerous methods, including those described herein, as well as any methods well-known in the art. For example, blood may be obtained through venipuncture performed at a peripheral vein (Lamy, B., et al. 2016. Frontiers in Microbiology, 7(697): 1-13; Dawson, S. 2014. Journal of Hospital Infection, 87(1), 1-10). To avoid contamination by skin bacteria and the generation of false positive blood cultures, the skin at the venipuncture site is thoroughly cleansed with an antibacterial solution. Studies demonstrate that skin preparation is more effectively performed with alcoholic iodine solutions or alcoholic chlorhexidine gluconate. Alcohol containing products are superior to aqueous solutions (Lamy, B., et al. 2016. Frontiers in Microbiology, 7(697): 1-13; Garcia, R. A., et al. 2015. American Journal of Infection Control, 43(11): 1222-1237). The bacterial contamination rate also diminishes if the antibacterial solution is also placed onto the blood culture bottle injection site (Lamy, B., et al. 2016. Frontiers in Microbiology, 7(697): 1-13). Once patient blood is injected sterilely into the blood culture bottle, the bottle is transported to the incubator as quickly as possible. Studies confirm that rapid incubation of blood culture bottles results in a higher yield of true positive blood cultures, and a lower contamination rate (Lamy, B., et al. 2016. Frontiers in Microbiology, 7(697): 1-13; Garcia, R. A., et al. 2015. American Journal of Infection Control, 43(11): 1222-1237; Kirn, T. J., et al. 2013. Clinical Microbiology and Infection, 19(6): 513-520; Reimer, L. G., et al. 1997. Clinical Microbiology Reviews, 10(3): 444-465).

It should be recognized by one skilled in the art that samples of whole blood are generally diluted with an anticoagulant upon drawing to prevent coagulation. Thus, in some embodiments, the present invention further comprises contacting the whole blood sample with one or more anticoagulant. In one embodiment, said contacting of the whole blood sample with one or more anticoagulant occurs prior to obtaining platelet rich plasma. It should be further noted that any art-recognized anticoagulant capable of preventing coagulation can be used with the methods of the present invention. For example, the anticoagulant may comprise one or more selected from the group consisting of: an ethylenediaminetetraacetic acid (EDTA) salt, a heparin salt, a citrate salt, an oxalate salt, acid citrate dextrose (ACD), argatroban, a low molecular weight heparin, Eliquis® (apixaban), Xarelto® (rivaroxaban), and dabigatran. In one embodiment, the anticoagulant comprises a heparin salt.

In one embodiment, the EDTA salt is one or more selected from the group consisting of: disodium EDTA, dipotassium EDTA, and tripotassium EDTA. In one embodiment, the heparin salt is one or more selected from the group consisting of: lithium heparin and sodium heparin. In one embodiment, the citrate salt is one or more selected from the group consisting of: monosodium citrate, disodium citrate, and trisodium citrate. In one embodiment, the oxalate salt is one or more selected from the group consisting of: ammonium oxalate and potassium oxalate.

In one embodiment, said ACD comprises a citrate salt, citric acid, and dextrose. In one embodiment, said ACD comprises a citrate salt at a concentration between 10 g/L and 25 g/L. In one embodiment, said ACD comprises a citrate salt at a concentration of about 22.0 g/L. In one embodiment, said ACD comprises a citrate salt at a concentration of about 13.2 g/L. In one embodiment, said ACD comprises citric acid at a concentration between 2 g/L and 12 g/L. In one embodiment, said ACD comprises citric acid at a concentration of about 8.0 g/L. In one embodiment, said ACD comprises citric acid at a concentration of about 4.8 g/L. In one embodiment, said ACD comprises dextrose at a concentration between 10 g/L and 30 g/L. In one embodiment, said ACD comprises dextrose at a concentration of about 14.7 g/L. In one embodiment, said ACD comprises dextrose at a concentration of about 24.5 g/L. In one embodiment, the citrate salt of said ACD comprises one or more selected from the group consisting of: monosodium citrate, disodium citrate, and trisodium citrate.

In one embodiment, the methods of the present invention exclude the use of any anticoagulant that may inhibit bacterial growth in culture. In one embodiment, the methods of the present invention exclude the use of an EDTA salt as an anticoagulant. While not being bound by scientific theory, it is believed that EDTA salt as an anticoagulant can inhibit bacterial growth and thus make the growth and detection of blood-borne bacteria less reliable.

It should also be recognized by one skilled the art that any well-known methods for preparing substantially enriched preparation of platelets in plasma (PRP) could be used with the present invention. PRP is obtained from whole blood or plasma by concentrating the platelets from the blood, most commonly using a centrifuge. In one embodiment, PRP is isolated using an EBA 21 Centrifuge (Andreas Hettich GmbH & Co. KG). While whole blood may contain about 95% red blood cells, about 5% platelets and less than 1% white blood cells, PRP may contain 95% platelets with 4% red blood cells and 1% white blood cells. Platelets are living but terminal cytoplasmic portions of marrow megakaryocytes. They have no nucleus for replication and will die off in 5-9 days. They adhere together to form a platelet plug at an injury site and actively extrude the growth factors involved in initiating wound healing. Platelets also play an active role in innate immunity.

In one embodiment, the platelet rich plasma (PRP) of the present invention is isolated by centrifugation at a speed of at least 2,000 rotations per minute (rpm). In one embodiment, the PRP is isolated by centrifugation at a speed up to and including 12,000 rpm. In one embodiment, the PRP is isolated by centrifugation at a speed up to and including 10,000 rpm. In one embodiment, the PRP is isolated by centrifugation at a speed up to and including 8,000 rpm. In one embodiment, the PRP is isolated by a centrifugation at a speed up to and including 6,000. In one embodiment, the PRP is isolated by centrifugation at a speed of up to 4,000 rpm. In one embodiment, the PRP is isolated by centrifugation at between 2,000 and 10,000 rpm. In one embodiment, the PRP is isolated by centrifugation at between 3,000 and 8,000 rpm. In one embodiment, the PRP is isolated by centrifugation at between 4,000 and 7,000 rpm. In one embodiment, the PRP is isolated by centrifugation at about 6,000 rpm. In one embodiment, the PRP is isolated by centrifugation at about 5,000 rpm. In one embodiment, the PRP is isolated by centrifugation at 6,000 rpm. In one embodiment, the PRP is isolated by centrifugation at 5,000 rpm. While not being bound by any particular theory, it is hypothesized that centrifugation at higher speeds improves availability of platelet-associated microbes for growth and subsequent detection, provided that such speed does not result in forces acting on the sample that ultimately result in loss of integrity or breakdown of the platelets themselves. High speed centrifugation would not be considered in the art to be optimal for preparing platelet rich plasma with the greatest number of intact, fully functioning platelets, as it can disrupt normal platelet function. A review of more than 100 studies involving isolation of human PRP found that in studies that reported centrifugation speed (59) and time (60), the average speed was 1,986±1,098 rpm (mean±standard deviation) and the average time was 11±4 minutes. Further, the maximum speed reported was 5,800 rpm (Chahla, J., et al. 2017. JBJS, 99(20), 1769-1779). However, higher speed centrifugation may be optimal for the release and subsequent availability of associated microbes. Additionally, higher speed centrifugation allows for isolation of PRP in a shorter time frame.

In some embodiments, a lower speed centrifugation may be advantageous. In one embodiment, centrifugation is performed at a speed of less than 3,000 rpm. In one embodiment, centrifugation is performed a speed of less than 2000 rpm. In one embodiment, centrifugation is performed at a speed of about 1,000, about 2,000, or about 3,000 rpm.

In one embodiment, the PRP is isolated by centrifugation for at least 1 minute. In one embodiment, the PRP is isolated by centrifugation for at least 2 minutes. In one embodiment, the PRP is isolated by centrifugation for up to and including 15 minutes. In one embodiment, the PRP is isolated by centrifugation for between 3 and 10 minutes. In one embodiment, the PRP is isolated by centrifugation for between 4 and 6 minutes. In one embodiment, the PRP is isolated by centrifugation for about 5 minutes. In one embodiment, the PRP is isolated by centrifugation for 5 minutes.

In one embodiment, the whole blood is pre-treated with an agent prior to centrifugation. In one embodiment, that agent promotes a change in the shape of native platelets. In one embodiment, the shape is altered from a native biconcave disc-like shape to spherical. In one embodiment, the agent comprises a chemotherapeutic drug. Chemotherapeutic drugs used for the purposes of the present invention include, but are not limited to, vinblastine, vincristine, Taxol® (paclitaxel), and Taxotere® (docetaxel). In one embodiment, the chemotherapeutic agent comprises taxol. In one embodiment, the agent comprises a gout drug. In one embodiment, the gout drug comprises colchicine. While not being bound by any particular theory, it is hypothesized that promoting the shape change of platelets improves the availability of encountered microbes adhered to the platelet surface for growth and subsequent detection. Accordingly, in some embodiments, the PRP is treated with an agent that promotes a change in the shape of native platelets.

The process of incubating blood or blood-derived samples to detect bacterial infection, as it relates to the present invention, can be performed using numerous methods, including those described herein, as well as any methods well-known in the art. For example, current blood culture media known in the art use carbohydrate substrates. Production of CO₂ by growing bacteria leads to their detection through a colorimetric or fluorescent sensor at the bottom of the bottle (Kim, T. J., et al. 2013. Clinical Microbiology and Infection, 19(6): 513-520). Investigators report that only slight improvements in the performance characteristics of culture media have been achieved in the last 15 years (Kim, T. J., et al. 2013. Clinical Microbiology and Infection, 19(6): 513-520).

In one embodiment of the present invention, incubation of the PRP occurs immediately after isolation. In one embodiment, incubation occurs less than 120 hours after isolation. In one embodiment, incubation occurs less than 96 hours after isolation. In one embodiment, incubation occurs less than 72 hours after isolation. In one embodiment, incubation occurs less than 48 hours after isolation. In one embodiment, incubation occurs less than 24 hours after isolation. In one embodiment, the incubation period lasts up to five days. In one embodiment, the incubation period is five days. In one embodiment, the incubation period is four days. In one embodiment, the incubation period is three days. In one embodiment, the incubation period is two days. In one embodiment, the incubation period is one day. In one embodiment, incubation occurs at a temperature between 33 and 37 degrees centigrade. In one embodiment, incubation occurs at a temperature between 34 and 36 degrees Centigrade. In one embodiment, incubation occurs at about 35 degrees Centigrade. When blood culture bottles are incubated, a false negative signal can occur. A false negative signal is defined as the absence of detected bacteria when, in fact, bacteria are present. Several parameters have been identified that raise the risk of false negative signals. The main predictor of false negativity is preincubation delay, in which excess time transpires between patient venipuncture and incubation (Reimer, L. G., et al. 1997. Clinical Microbiology Reviews, 10(3): 444-465). False negativity can also depend on the temperature at which the bottle was kept prior to incubation, type of bacteria and the type of bottle used. Studies reveal that a false negative signal was greater when blood cultures were kept at <35 degrees Centigrade at preincubation, time from venipuncture to incubation was >24 hours, particularly in the presence of Pseudomonas or Streptococcus species (Reimer, L. G., et al. 1997. Clinical Microbiology Reviews, 10(3): 444-465). Bacteria growing in blood culture bottles kept at relatively high temperatures, (>35 degrees centigrade) have been shown to already have reached their stationary phase of growth so that their presence can no longer be detected by the current methodology employed in automated incubation systems (Seegmüller, I., et al. 2004. Journal of Medical Microbiology, 53(9): 869-874). On the other hand, a false positive signal can occur, defined as the detection of bacteria when, in fact, none are present. This false positive signal occurs in less than 1% of cultured media bottles, and can occur as the result of overfilled bottles, elevated patient white blood cell count, or errors in incubation (Reimer, L. G., et al. 1997. Clinical Microbiology Reviews, 10(3): 444-465).

In one embodiment of the present invention, the volume of blood required for bacterial detection is substantially less than is recommended for blood cultures utilizing whole blood. In one embodiment, said volume of blood drawn comprises a volume that is small enough to avoid dangerous physiologic stress to the patient. In one embodiment, said volume is less than 100 milliliters. In one embodiment, said volume is less than 60 milliliters. In one embodiment, said volume is less than 40 milliliters. In one embodiment, said volume is less than 20 milliliters. In one embodiment, said volume is between 1 and 10 milliliters. In on embodiment, said volume is between 2 and 5 milliliters. In one embodiment, said volume is about 3 milliliters. In one embodiment, said volume is 2.9 milliliters. Numerous studies have confirmed that blood stream infections can be more reliably detected when traditional venipuncture volumes are larger and more frequent (Jonsson, B., et al.1993. APMIS, 101(7-12): 595-601; Mermel, L. A., et al. 1993. Annals of Internal Medicine, 119(4): 270-272; Cockerill III, F. R., et al. 2004. Clinical Infectious Diseases, 38(12): 1724-1730; Patel, R., et al. 2011. Journal of Clinical Microbiology, 49(12): 4047-4051; Bouza, E., et al. 2007. Journal of Clinical Microbiology, 45(9): 2765-2769; Lee, A., et al. 2007. Journal of Clinical Microbiology, 45(11): 3546-3548). Blood culture positivity increases in a linear fashion as the volume of blood sampled increases from 20 to 40 to 60m1 (Mermel, L. A., et al. 1993. Annals of Internal Medicine, 119(4): 270-272). Nonetheless, the sensitivity of blood cultures performed in the traditional manner in patients with sepsis remains disturbingly low, in the range of just 32% to 71%, with most studies reporting a sensitivity of under 50%. (Alam, N., et al. 2018. The Lancet Respiratory Medicine, 6(1): 40-50; Kumar, A., et al. 2009. Chest, 136(5): 1237-1248; Bernard, G. R., et al. 2001. Critical Care Medicine, 9(11): 2051-2059; Phua, J., et al. 2013. Critical Care, 17(202): 1-12; Brooks, D., et al. 2016. American Journal of Infection Control, 44(11): 1291-1295). Because the concentration of circulating bacteria is quite low, in the range of 0.01 to 1.0 colony forming units per mL, Jonnson et al have demonstrated that 30 cc of blood must be cultured to maximize detection rates (Jonsson, B., et al.1993. APMIS, 101(7-12): 595-601). Professional societies have therefore made recommendations on the frequency of venipuncture and volume of blood obtained to optimize detection of blood stream infection. The American College of Critical Care Medicine and the Infectious Disease Society of America have jointly recommended that 3 to 4 blood culture sets be performed with 20 to 30 ml per culture set (O'Grady, N. P., et al. 2008. Critical Care Medicine, 36(4): 1330-1349). The joint recommendations of the American Society of Microbiology and the Infectious Disease Society of America is to draw 2 to 4 sets of blood cultures with 20 to 30 ml of blood per set. The UK Standards for Microbiology recommends 4 bottles equaling 2 sets of 20 ml of blood per set (Standards Unit, Public Health England. 2014. UK Standards for Microbiology Investigations, B37(8.2): 1-55). The French Society of Microbiology recommends 4 to 6 bottles of blood, representing 2 to 3 sets (French Society of Microbiology. 2018. Standards in Medical Microbiology. 6). Generally, the total amount of blood recommended by these societies is quite high, and these volumes are poorly tolerated by severely ill patients who are already anemic and hypotensive as a consequence of their septic state. The high volume of blood recommended by the above societies could easily serve to exacerbate the underlying pathophysiology of the septic patient. A tremendous advantage of utilizing PRP is that the volume of blood required is dramatically less, just 2.9 ml. In fact, this minute amount of blood is already available in all patients with suspected blood stream infection because all such patients will have already had a sample for complete blood counts (CBC) drawn upon presentation—just 2.9 ml of whole blood injected into a small tube containing 0.1 ml of sodium citrate as an anticoagulant. This CBC sample is generally drawn “stat” upon patient presentation, is always transmitted to the Hematology laboratory promptly for analysis where PRP can be rapidly and easily prepared by centrifugation according to the methods of the invention for immediate sterile transfer into a blood culture bottle, then immediately incubated. Just 2.9 ml of whole blood entails substantially less physiologic risk to the septic patient, unlike the much higher volumes demanded by traditional blood culture methods.

In one embodiment of the present invention, smaller volumes reduce contamination as compared to blood cultures utilizing whole blood. In one embodiment, said reduction in contamination reduces false positive detection of blood-borne microbes as compared to blood cultures utilizing whole blood. In addition to reducing physiologic stress, the small amount of blood required to create PRP and the single venipuncture required also serves to reduce the risk of false positive blood cultures via contamination (Lamy, B., et al. 2016. Frontiers in Microbiology, 7(697): 1-13; Garcia, R. A., et al. 2015. American Journal of Infection Control, 43(11): 1222-1237; Kim, T. J., et al. 2013. Clinical Microbiology and Infection, 19(6): 513-520; Reimer, L. G., et al. 1997. Clinical Microbiology Reviews, 10(3): 444-465), resulting in significant savings in cost of care, and patient morbidity resulting from the inappropriate use of antibiotics and diagnostic testing (Lamy, B., et al. 2016. Frontiers in Microbiology, 7(697): 1-13; Garcia, R. A., et al. 2015. American Journal of Infection Control, 43(11): 1222-1237; Kim, T. J., et al. 2013. Clinical Microbiology and Infection, 19(6): 513-520; Reimer, L. G., et al. 1997. Clinical Microbiology Reviews, 10(3): 444-465). The higher yield provided by culturing with PRP as compared with whole blood would also inform the selection of appropriate antibiotics, thus optimizing the patient's chance of survival, reducing drug related morbidities, and reducing hospital length of stay and overall costs of care (Kumar, A., et al. 2009. Chest, 136(5): 1237-1248).

### Sepsis

In one embodiment, the present invention comprises an improved method for detecting blood-borne microbes in a patient exhibiting symptoms of sepsis. Symptoms of sepsis include, but are not limited to, high body temperature, low body temperature, chills, shivering, accelerated heart rate, accelerated breathing, shortness of breath, dizziness, confusion, disorientation, nausea, vomiting, diarrhea, slurred speech, and severe muscle pain.

Of patients hospitalized in an intensive care unit (ICU) who have an infection, 82% have sepsis. Sepsis may be defined as an infection-induced syndrome involving two or more of the following features of systemic inflammation: fever or hypothermia, leukocytosis or leukopenia, tachycardia, and tachypnea or a supranormal minute ventilation. Sepsis may also be defined by the presence of any of the following ICD-9-CM codes: 038 (septicemia), 020.0 (septicemic), 790.7 (bacteremia), 117.9 (disseminated fungal infection), 112.5 (disseminated Candida infection), and 112.81 (disseminated fungal endocarditis).

Sepsis is diagnosed either by clinical criteria or by culture of microorganisms from the blood of patients suspected of having sepsis plus the presence of features of systemic inflammation. Culturing some microorganisms can be tedious and time-consuming and may provide a high rate of false negatives using methods well-15 known in the art. Bloodstream infection is diagnosed by identification of microorganisms in blood specimens from a patient suspected of having sepsis after 24 to 72 hours of laboratory culture. Currently, gram-positive bacteria account for 52% of cases of sepsis, gram-negative bacteria account for 38%, polymicrobial infections for 5%, anaerobes for 1%, and fungi for 5%. For each class of infection listed, there are several different types of microorganisms that can cause sepsis. The high rate of false negative microbiologic cultures leads frequently today to empiric treatment for sepsis in the absence of definitive diagnosis. Infection at many different sites can result in sepsis. The most common sites of infection in patients with sepsis are lung, gut, urinary tract, and primary blood stream site of infection. Since sepsis can be caused infection with microorganisms at many different sites, sepsis is a very heterogeneous disease. The heterogeneity of sepsis increases the difficulty in devising a diagnostic test.

In one embodiment, the present invention comprises an improved method for detecting blood-borne microbes in a patient exhibiting symptoms of sepsis to diagnose a microbial infection and appropriately determine a course of treatment. While studies have been conducted to assess bacterial growth in donated PRP preparations for therapeutic use, it should be appreciated by one in the art that the motivations for such studies differ distinctly from that of the present invention. For example, the motivation to assess bacterial growth in donor-derived PRP is to ensure no contamination prior to administering said PRP to a patient in need thereof. By contrast, the motivation to detect bacterial growth in PRP of the present invention is to determine the nature of bacterial infection leading to sepsis, and to accurately determine an appropriate course of treatment for said patient. Further, one of skill in the art will appreciate that methods employed by previous studies are distinctly different from those of the present invention. For example, bacterial growth has been assessed from at least 100 mL of donor-derived PRP for therapeutic use, but in one embodiment, less than 20 mL of PRP is required according to the methods of the present invention. Additionally, donor-derived PRP for therapeutic use is typically stored for several days prior to an assessment of bacterial growth, whereas, in one embodiment of the present invention, PRP is assessed immediately. Finally, donor-derived PRP for therapeutic use is derived from healthy donors, whereas PRP of the present invention, in one embodiment, is derived from patients exhibiting symptoms of sepsis.

### Pathogenic Bacteria

In one embodiment, the blood-borne microbe to be detected by the methods of the present invention is pathogenic. In one embodiment, the pathogenic microbe is a bacterium.

Examples of pathogenic bacteria include, but are not limited to staphylococcus (for example, Staphylococcus aureus, Staphylococcus epidermidis, or Staphylococcus saprophyticus), streptococcus (for example, Streptococcus pyogenes, Streptococcus pneumoniae, or Streptococcus agalactiae), enterococcus (for example, Enterococcus faecalis, or Enterococcus faecium), corynebacteria species (for example, Corynebacterium diptheriae), bacillus (for example, Bacillus anthracis), listeria (for example, Listeria monocytogenes), Clostridium species (for example, Clostridium perfringens, Clostridium tetanus, Clostridium botulinum, Clostridium difficile), Neisseria species (for example, Neisseria meningitidis, or Neisseria gonorrhoeae), E. coli, Shigella species, Salmonella species, Yersinia species (for example, Yersinia pestis, Yersinia pseudotuberculosis, or Yersinia enterocolitica), Vibrio cholerae, Campylobacter species (for example, Campylobacter jejuni or Campylobacter fetus), Helicobacter pylori, pseudomonas (for example, Pseudomonas aeruginosa or Pseudomonas mallei), Haemophilus influenzae, Bordetella pertussis, Mycoplasma pneumoniae, Ureaplasma urealyticum, Legionella pneumophila, Treponema pallidum, Leptospira interrogans, Borrelia burgdoferi, mycobacteria (for example, Mycobacterium tuberculosis), Mycobacterium leprae, Actinomyces species, Nocardia species, chlamydia (for example, Chlamydia psittaci, Chlamydia trachomatis, or Chlamydia pneumoniae), Rickettsia (for example, Rickettsia ricketsii, Rickettsia prowazekii or Rickettsia akari), brucella (for example, Brucella abortus, Brucella melitensis, or Brucella suis), Proteus mirabilis, serratia (for example, Serratia marcescens, Serratia liquefaciens, and Serratia ficaria), Enterobacter clocae, Acetinobacter anitratus, Klebsiella pneumoniae and Francisella tularensis.

### Detection

In one embodiment, the rate of true positive detection of blood-borne microbes of the present invention is greater than that of blood cultures utilizing whole blood. In one embodiment, sensitivity of detection of blood-borne microbes of the present invention is greater than that of blood cultures utilizing whole blood. In one embodiment, the time to detection of blood-borne microbes of the present invention is less than that of blood cultures utilizing whole blood.

It will be appreciated by one skilled in the art that any methods of determining the presence of a blood-borne microbe can be used with the present invention. For example, common methods comprise continuous-monitoring automated blood culture systems including, but not limited to, Bactec (Beckton-Dickinson, USA) and BactAlert/Virtuo systems (bioMérieux, France).

Further, it will be appreciated by one skilled in the art that any methods of detecting the specific nature of a blood-borne microbe can be used with the present invention. Examples of techniques that can be used include, but are not limited to, sequencing, immunoassays, mass spectrometry (MS), and culturing. In some embodiments, the microbe is detected by sequencing. Examples of sequencing useful in the present invention include, but are not limited to, fragment analysis, first generation next generation sequencing (NGS), Sanger sequencing, second generation NGS, shotgun sequencing, pyrosequencing, bridge amplification sequencing, reversible terminator sequencing, sequencing-by-ligation, ion semiconductor sequencing, sequencing by synthesis, combinatorial probe anchor synthesis, emulsion PCR, complementary metal oxide semiconductor sequencing, third generation NGS, nanopore sequencing, single-molecule sequencing, single-molecule real-time sequencing, massively parallel signature sequencing, polony sequencing, microfluidic sequencing, and metagenomic NGS.

In some embodiments, the microbe is detected by one or more immunoassays. Immunoassays that may be useful for determining the nature of a blood-borne microbe include, but are not limited to, monoclonal antibody detection, enzyme-linked immunosorbent assay (ELISA), direct ELISA, indirect ELISA, sandwich ELISA, competitive ELISA, colorimetric ELISA, chemiluminescent ELISA, Radioimmunoassay (MA), Western blot, Southern blot, and Northern blot.

In some embodiments, the microbe is detected by mass spectrometric methods. Mass spectrometric methods include, but are not limited to, protein sequencing, nucleotide sequencing, and metabolomic analysis.

In some embodiments, the microbe is detected by a combination of methods. For example, Western blotting may be followed by gel excision and protein sequencing by mass spectrometry. In some embodiments, these methods are additionally be combined with other preparative techniques including, but not limited to, capillary electrophoresis, gel electrophoresis, liquid chromatography, gas chromatography, affinity chromatography, size-exclusion chromatography, and ion exchange chromatography. For example, a bacterium can be detected using liquid chromatography-mass spectrometry (LC-MS) to identify a bacterium's unique molecular fingerprint. Systems employed for such detection include, but are not limited to, the Bruker-Maldie Biotyper CA System.

It will be appreciated by those in the art that in some embodiments, the methods of detection described herein are applicable not only to the detection of intact living microbes, but also to the detection of microbe-derived molecules. Examples of microbe-derived molecules that can be detected include, but are not limited to, DNA molecules, RNA molecules, proteins, and fragments thereof, as well as small molecule metabolites. Accordingly, in some embodiments the method allows for detecting the presence of microbes in a platelet-rich plasma sample even if the microbe cannot be cultured by conventional means.

### Antibiotic Therapy

In one embodiment, the present invention relates to a method of treating a patient with a microbial infection, comprising the steps of: 1) detecting the presence of one or more blood-borne microbes, 2) selecting one or more antibiotic specific to one or more blood-borne microbe detected in the PRP of said patient; and 3) treating said patient with a therapeutically effective amount of one or more antibiotic specific to one or more blood-borne microbe detected in the PRP of said patient. In one embodiment, said antibiotic selection results in fewer non-specific antibiotics being used to treat said patient as compared to antibiotic selection based upon blood cultures utilizing whole blood. In one embodiment said antibiotic selection reduces the risk of drug-reacted morbidities as compared to antibiotic selection based upon blood cultures utilizing whole blood. In one embodiment, said reduction in the risk of drug-reacted morbidities increases patient survival rates as compared to antibiotic selection based upon blood cultures utilizing whole blood.

The antibiotics of the present invention include both natural antibiotics, chemical substances produced by microorganisms such as bacteria and fungi, semisynthetic antibiotics, which are derivatives obtained through structural changes of natural antibiotics, and chemically synthesized synthetic antibiotics (antimicrobial agents).

The antibiotics of the present invention include, but are not limited to, aminoglycosides, penicillins, cephalosporins, tetracycline, macrolides, streptogramins, glycoconjugates, peptides, flavophospholipols, polyethers, phenicols, lincosamides, a rifamycins, polyphosphates, polyenes, sulfonamides, benzylpyridines , quinolones, fluoroquinolones, and nitrofuran antibiotics.

Examples of the aminoglycoside antibiotics include, but are not limited to, gentamycin, kanamycin, ribostamycin, tobramycin, sisomycin, astromycin, isepamycin, abcacin, dibecasin, spectinomycin, nethylamycin, micronomycin, streptomycin, dihydrostreptomycin, apramycin, desmomycin, hygromycin, amikacin, and neomycin.

Penicillin antibiotics include, but are not limited to, penicillin, benzylpenicillin, oxacillin, clock factin, dicloxiline, fluclock truein, napusylin, ampicillin, amoxicillin, carbenicillin, ticacillin, piperacillin.

Cephalosporin-based antibiotics include, but are not limited to, cephalothin, cepharolin, cephaloridine, cephalexin, cephalosyl, cephalosporin, cephalosporin, cepharillin, cephadoxil, rosemary, cell ferazone, cephamethazole, cell taksim, ceftiofur, and the like.

Tetracycline antibiotics include, but are not limited to, chloretracycline, oxytetracycline, tetracycline, minocycline, and doxycycline.

Macrolide antibiotics include, but are not limited to, erythromycin, chitamycin, spiramycin, oleandomycin, irradiamycin, sedecamycin, tyrosine, mikamycin and roxithromycin.

Streptogramin antibiotics include, but are not limited to, virginiamycin, quinupristin/dalfopristin, NXL, 103 and pristinamycin.

Glycopeptide antibiotics include, but are not limited to, avoparcin, vancomycin, teicoplanin, telavancin, ramoplanin, decaplanin, corbomycin, and complestatin.

Peptide-based antibiotics include, but are not limited to, polymyxins (e.g., colistin, polymyxin B, polymyxins E, etc.), cyclic peptides (e.g., bacitracin, enramycin, gramicidin S etc), and natural antimicrobial peptides (e.g., defensins, bombinins, cecrophins, etc.).

Flavophospholipol-based antibiotics include, but are not limited to, Burma Bacillus, Macabomycin, Flavomycin, Bamebermycin and Quebemycin.

Polyether antibiotics include, but are not limited to, monensin, salinomycin, lasaroside, lasalocid, narasin, nigericin, lenoremycin, carriomycin, septomycin, carriomycin, alborixin, calcimycin, lysocellin, lonomycin and narycinaminodalamicin.

Phenicol antibiotics include, but are not limited to, chloramphenicol, thiamphenicol, and florfenicol.

Lincosamide antibiotics include, but are not limited to, lincomycin, pirlimycin and clindamycin.

The rifamycin antibiotics include, but are not limited to, rifampicin, rifabutin, rifapentine, rifalazil, rifaximin, and lymphampicin.

Polyene antibiotics include, but are not limited to, nystatin, pimaricin, pentamycin, amphotericin B, tricomycin, and candicidin.

Sulfonamide antibiotics include, but are not limited to, sulfacetamide sulfadiazine, sulfadimidine, sulfafurazole, sulfisomidine, sulfapyridine, sulfamethoxazole, sulfamoxole, sulfanitran, sulfadimethoxine, sulfamethoxypyridazine, sulfametoxydiazine, sulfadoxine, sulfametopyrazine, and terephtyl.

Benzylpyridine antibiotics include, but are not limited to, trimethoprim, ometoprim, and tetroxoprim.

Quinolone antibiotics include, but are not limited to, nadidic acid, nalidixic acid, oxolinic acid, cinoxacin, and acrothosin.

Fluoroquinolone antibiotics include, but are not limited to, ciprofloxacin, levofloxacin, gatifloxacin, moxifloxacin, ofloxacin, norfloxacin, flumequeen, sipurosacin, enosacin, fueroxacin, and magofloxacin.

Nitrofuran antibiotics include, but are not limited to, difurazone, furazolidone, nifurfoline, nifuroxazide, nifurquinazol, nifurtoinol, nifurzide, nitrofural, nitrofurantoin, ranbezolid, furaltadone, furazidine, furylfuramide, nifuratel, nifurtimox, nitrophenyl, and nitroprazone.

### Negative Diagnosis

In one embodiment, detection of the presence of one or more microbes according to the methods of the present invention excludes the diagnosis of a viral infection that presents with similar symptoms. Viruses that can cause infections resulting in symptoms that could be misdiagnosed as bacterial infection include, but are not limited to, Epstein-Barr virus, Hepatitis B virus, Hepatitis C virus, Cytomelagovirus, Rhinovirus, Echovirus, and Ebola virus.

In one embodiment, said viral infection is severe acute respiratory syndrome coronavirus 2 (SARS-CoV-2) infection. SARS-CoV-2 infection shares many clinical similarities with sepsis and represents a serious global health burden. While not being bound by any particular theory, it is hypothesized that the methods of the present invention represent a significant improvement in the ability to exclude viral infection, including COVID-19, as a likely cause of sepsis-like symptoms. By allowing more rapid and sensitive cultures while requiring less blood, the present invention represents a significant advancement in the ability to triage and manage patients under normal circumstances and during a global crisis, such as the COVID-19 pandemic.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Detection of Blood-Borne Bacteria from Platelet Rich Plasma

Although blood platelets play a major role in coagulation and hemostasis, over the past decade overwhelming evidence has confirmed that platelets play a major role in human immune defense, especially in the bodies' response to bacterial infection (Assinger, A., et al. 2019. Frontiers in Immunology, 10(1687): 1-19; Palankar, R., et al. 2018. Journal of Thrombosis and Haemostasis, 16(6): 1187-1197; Claushuis, T. A., et al. 2018. Blood, 131(8): 864-876; Wang, Z., et al. 2016. Blood Coagulation & Fibrinolysis, 27(6): 667-672; Soares, A. C., et al. 2002. British Journal of Pharmacology, 137(5): 621-628; Krauel, K., et al. 2012. Blood, 120(16): 3345-3352; Moriarty, R. D., et al. 2016. Journal of Thrombosis and Haemostasis, 14(4): 797-806; Assinger, A., et al. 2012. Thrombosis Research, 130(3): e73-e78); Tohidnezhad, M., et al. 2012. Platelets, 23(3): 217-223; Watson, C. N., et al. 2016. Platelets, 27(6): 535-540; de Stoppelaar, S. F., et al. 2015. Journal of Thrombosis and Haemostasis, 13(9): 1709-1720; De Stoppelaar, S. F., et al. 2015. Journal of Thrombosis and Haemostasis, 13(6): 1128-1138). In response to a vast variety of bacteria that are pathologic to humans, platelets coordinate with white blood cells and endothelial cells that line blood vessels to provide a powerful antibacterial response (Wang, Z., et al. 2016. Blood Coagulation & Fibrinolysis, 27(6): 667-672; De Stoppelaar, S. F., et al. 2015. Journal of Thrombosis and Haemostasis, 13(6): 1128-1138). Upon infection, platelet factor 4 is released from internal platelet granules, binds onto a variety of bacterial surfaces, and thus changes the 3 dimensional conformation of anionic proteins so as to render bacteria more susceptible to opsonization ingestion) by white blood cells (Assinger, A., et al. 2019. Frontiers in Immunology, 10(1687): 1-19; Palankar, R., et al. 2018. Journal of Thrombosis and Haemostasis, 16(6): 1187-1197; Soares, A. C., et al. 2002. British Journal of Pharmacology, 137(5): 621-628; Krauel, K., et al. 2012. Blood, 120(16): 3345-3352; Tohidnezhad, M., et al. 2012. Platelets, 23(3): 217-223). Platelets interact directly with innate immune cells, exert important immunomodulatory effects during active bacterial infection and promote white blood cell (WBC) migration to sites of infection (Wong, C. H., et al. 2013. Nature Immunology, 14(8): 785-792; Wang, Z., et al. 2016. Blood Coagulation & Fibrinolysis, 27(6): 667-672). Further, platelets assist monocytes in differentiating into macrophages, a key cell in bacterial phagocytosis. In fact, platelet surface Toll—like receptors play a key role in regulating bacterial phagocytosis (Assinger, A., et al. 2012. Thrombosis Research, 130(3): e73-e78). A major platelet surface protein, FcγR2A (CD32A), promotes immune complex-mediated platelet activation for killing of opsonized bacteria (Moriarty, R. D., et al. 2016. Journal of Thrombosis and Haemostasis, 14(4): 797-806; Watson, C. N., et al. 2016. Platelets, 27(6): 535-540). Thus, platelets seem to “know” which precise bacteria are involved in the genesis of sepsis.

The present invention is based on the ability of platelets to recognize the harmful bacteria that cause infections and can lead to sepsis. Reduction to practice experiments have tested the hypothesis and confirmed that the blood platelet compartment, consisting of trillions of platelets circulating in blood, is a more reliable source of precise microbiologic information when used to perform blood cultures than performing cultures with whole blood using traditional techniques. Furthermore, the present invention teaches the detailed process to provide a significant improvement in the accuracy of teaching. This teaching is a useful improvement over the well-known process that has been in use for decades. Platelets in very large quantities are isolated by centrifugation so as to create platelet rich plasma. Injection of platelet rich plasma into standard blood culture tubes improves the sensitivity of blood cultures in detecting blood stream infection. The higher detection rate of circulating bacteria in blood made possible by the present invention may result in the informed selection of appropriate antibiotics in treatment dependent upon the precise bacterium so identified. This improves patient mortality and morbidity, decreases time spent in the Intensive Care Unit, avoids the expense and toxicity of the “shotgun” antibiotic approach and decreases overall health care costs (Kumar, A., et al. 2009. Chest, 136(5): 1237-1248; Phua, J., et al. 2013. Critical Care, 17(202): 1-12).

The Methods of the present Example are now described

The present invention, as previously conceived, was reduced to practice in a real-life clinical study at a medical institution. This research was approved by the Institutional Review Board. Initially, the study included 48 patients with suspected severe bacterial infection. Patients were recruited in the emergency room, ICU and medical wards. All of the patients had already had traditional blood cultures performed. After informed consent was obtained, the patient's Complete Blood Count (CBC) tube was identified in the Hematology Laboratory and centrifuged to create platelet rich plasma (PRP). The centrifugation was performed in the Hettich Model EBA 21 centrifuge. Centrifugation parameters were varied over the course of the study. Speeds were typically between 1,000 and 6,000 rpm, and centrifugation times were between 1 min and 5 min. In some cases, the centrifugation was performed in the Hettich Model EBA 21 centrifuge at 6,000 rpm for 5 minutes. In other cases, the centrifugation was performed at 2,000 rpm for 2 min. PRP was then sterilely pipetted into identical blood culture bottles. The volume of platelet rich plasma removed from the CBC tube varied between 0.8 and 1.8 mL. The PRP-containing blood culture bottle was then immediately taken to the institution's microbiology lab and placed in the incubator maintained between 34-36 degrees Centigrade. Both traditional and PRP blood cultures were incubated in the same manner. Traditional blood cultures were marked and identified in the usual manner at our institution including patient name, date of birth, and patient location and unique patient identification number. PRP blood cultures were identified by the patient's initials and unique study number, and patient confidentially was assured. The precise times that traditional and PRP blood cultures were performed were recorded. The routine assessment of blood culture results was conducted exclusively by the institution's microbiology staff. This assessment was conducted daily for a maximum of 5 days. Immediately upon detection of a positive blood culture, a small aliquot of fluid was aspirated from the culture bottle and plated onto both blood agar and chocolate agar plates so as to grow bacterial colonies. In approximately 24 hours, a tiny sample of the developing colony was removed from the plates and plated onto a glass slide, and a Gram stain was performed to assist in bacterial identification. Further, a tiny amount of the colony was placed onto a card and placed into a Bruker-Maldie Biotyper CA System which uses mass spectrometry to precisely identify the bacterium at hand through its molecular fingerprint. Results of traditional blood cultures were recorded in the patient's Medical Record so as to be made available to treating clinicians. Results of PRP blood cultures were only made known to the inventor, and not communicated to clinical staff as per the rules provided by our IRB. Thus, no clinical decisions were made on the basis of the results of PRP blood cultures.

The Results of the present Example are now described.

Confirmation of the benefits of blood cultures prepared from platelet rich plasma relative to traditional blood cultures were made early in the study when PRP blood cultures grew Enterococcus faecalis in an 80-year-old woman while her traditional blood culture prepared from whole blood grew nothing. This result is significant because the conventional whole blood culture technique failed to reveal the serious bacterium in the patient. In contrast, using the teaching of the present invention clearly identified the infectious bacterium. Further, the rapidity with which blood stream infection is detected by utilizing PRP in blood culturing is significantly greater than culturing with whole blood. While CBC samples were routinely obtained >12 hours after whole blood cultures had begun incubating, PRP samples isolated from said CBC samples were capable of detecting infection within the same time frame following the blood draw. More rapid detection of blood borne bacteria enables the more rapid administration of effective antibiotics, specific for the bacterium detected, in these very sick patients, which has been demonstrated to reduce patient mortality in sepsis (Kumar, A., et al. 2009. Chest, 136(5): 1237-1248). The utility of this teaching is significant in its accuracy and it may save lives, reduce patient morbidity and reduce hospital length of stay and health care costs (Kumar, A., et al. 2009. Chest, 136(5): 1237-1248; Phua, J., et al. 2013. Critical Care, 17(202): 1-12). This observation supports the conclusion that preparing blood cultures from PRP is an improvement over cultures traditionally prepared from whole blood in terms of the rate and sensitivity of detection and the volumes of blood required. The present invention is a significant improvement in blood culture technique and has the capacity to revolutionize this important field in Microbiology and Infectious diseases.

### Example 2: Detection of Blood-Borne Bacteria from PRP: a Case Study

The case study was expanded to 100 patients suspected of microbial infection and was carried out at Abington Memorial Hospital (AMH). AMH has a state-of-the-art Microbiology Lab. The cultures were performed using the BACTEC system, which is Becton Dickinson's art-recognized gold-standard automated blood culture system for bacterial detection.

Of the 100 patients examined, for which traditional whole blood culture was compared to PRP culture for bacterial detection, two patients demonstrated the presence of bacteria in blood when blood cultures were conducted with plate rich plasma, while traditional blood culture tests performed at AMH were negative.

Patient #2 was a 79-year-old woman, now deceased. She was admitted from a nursing home after an unwitnessed fall out of bed. She reported cough for 1 month and mild shortness of breath. Also, chills at the nursing home in recent days. She had completed Speech Pathology interventions 2 to 3 weeks earlier due to her aspiration risk and was on a modified diet. CT scan of chest showed consolidation pneumonia in the right upper lobe, right middle lobe, and left pper lobe consistent with polylobar pneumonia. Pulmonary nodules were also detected. Past Medical History showed irritable bowel syndrome, osteoporosis, chronic kidney disease, bipolar disease, and hypothyroidism. On admission she was hypoxic with an 02 saturation of just 94% on 2 liters of 02 per nasal cannula. Rectal temperature was 94.5 degrees. Respiratory rate was 16 breaths per minute and pulse was 66 beats per minute. Physical Exam was remarkable for diminished breast sounds in both lungs. Lactic acid was normal. Notably, platelet count was low at 103,000 platelets per microliter (normal is greater than 140,000 platelets per microliter) consistent with sepsis. White blood cell (WBC) counts were elevated at 11,800 per microliter with elevated neutrophil counts at 10,100 per microliter. Urinalysis was negative. Patient was treated with Cefipime and metronidazole and discharged on the fifth hospital day. The Internal Medicine team was unable to simplify antibiotic therapy because the state-of-the-art hospital cultures were negative. Therefore, she received broad spectrum antibiotics. Enterococcus faecalis was detected from cultures using PRP. The patient was successfully discharged from the hospital, and passed away months later from unrelated conditions.

Patient #62 was an 82-year-old man, now deceased. He was admitted from a nursing home with nausea, vomiting and diarrhea with low grade fever for 3 days. He had had a heart transplant more than 10 years earlier and was on immunosuppressive treatment with Tacrolimus. Past Medical History showed a history of bladder cancer treated with chemotherapy and bladder resection, placement of an illegal conduit, hypertension, Type 2 diabetes mellitus, obstructive sleep apnea, and hypothyroidism. He clearly had septic shock upon admission: Blood pressure was just 72/45, O₂ saturation was just 86% on room air, and lactic acid was elevated at 2.2 millimoles per liter. Physical examination was remarkable for lethargy and chest exam with reduced breath sounds. He showed elevated WBC counts at 11,300 per microliter with elevated neutrophil counts at 9,180 per microliter. Hemoglobin was low at 9.2 g/dL, and platelet counts were low normal at 162,000 per microliter. Blood tests revealed acute renal failure with creatinine levels of 1.61 mg/dL. AMH blood cultures were negative, while PRP cultures grew Burkholderia capecia, a known human pathogen. Patient was made DNR by his family and shortly after admission was discharged to home hospice.

In conclusion, of 100 patients examined in this study, traditional whole blood cultures were unable to detect established bacterial pathogens while PRP cultures were successful. Patient #2 had Enterococcus faecalis infection while Patient #62 had Burkholderia capecia infection as assessed from PRP blood cultures. Notably, whole blood samples prior to obtaining PRP of the present study were anticoagulated with sodium EDTA, which has been shown to have an inhibitory effect on bacterial growth (Banin et al., Appl Environ Microbiol, 2006 March;72(3):2064-9; Finnegan, et al., Adv Wound Care (New Rochelle), 2015 Jul. 1; 4(7): 415-421; Umerska, et al., Biomolecules 2018 Oct. 23;8(4):122). While not being bound by scientific theory, it is believed that anticoagulants that are not known to inhibit bacterial growth, such as one or more heparin salt, may be able to detect bacterial growth with even greater sensitivity. Thus, the present example demonstrates that cultures of PRP can detect bacterial infection in circumstances where traditional whole blood cultures fail. Notably, heparin is not shown to have a similar antibacterial effect (Zappala, et al.; Crit Care Resusc 2007 June;9(2):157-60).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of detecting one or more blood-borne microbe in a subject at risk of microbial infection, comprising the steps of:
obtaining platelet rich plasma (PRP) from a sample of whole blood drawn from said subject;
incubating the PRP at a temperature greater than room temperature for up to 5 days, wherein said incubating step occurs less than 24 hours after said obtaining step; and
detecting the presence of one or more blood-borne microbes in said PRP.

2. The method of claim 1, wherein one or more of said blood-borne microbe comprises a pathogenic microbe.

3. The method of claim 2, wherein the pathogenic microbe comprises a bacterium.

4. The method of claim 1, wherein said subject exhibits symptoms of sepsis.

5. The method of claim 4, wherein said subject comprises a human.

6. The method of claim 1, further comprising contacting said whole blood sample with one or more anticoagulant prior to obtaining PRP.

7. The method of claim 6, wherein said anticoagulant comprises one or more selected from the group consisting of: an ethylenediaminetetraacetic acid (EDTA) salt, a heparin salt, a citrate salt, an oxalate salt, acid citrate dextrose (ACD), argatroban, a low molecular weight heparin, Eliquis® (apixaban), Xarelto® (rivaroxaban), and dabigatran.

8. The method of claim 1, wherein the volume of said whole blood sample from said subject comprises a volume that is substantially less than is recommended for blood cultures utilizing whole blood.

9. The method of claim 6, wherein said sample of whole blood drawn is less than 20 milliliters in volume.

10. The method of claim 7, wherein said volume reduces contamination and false positive detection of blood-borne microbes as compared to blood cultures utilizing whole blood.

11. The method of claim 1, further comprising the step of contacting said whole blood sample with an agent that induces a change in the shape of platelets from biconcave discs to spherical.

12. The method of claim 1, wherein said detecting step comprises mass spectrometric analysis.

13. The method of claim 1, wherein the rate of true positive detection of blood-borne microbes is greater than that of blood cultures utilizing whole blood.

14. The method of claim 1, wherein the sensitivity of detection of blood-borne microbes is greater than that of blood cultures utilizing whole blood.

15. The method of claim 1, wherein the time to detection of blood-borne microbes is less than that of blood cultures utilizing whole blood.

16. A method of selecting a therapeutic specific to one or more blood-borne microbe in a subject at risk of microbial infection, comprising the steps of:
obtaining platelet rich plasma (PRP) from a sample of whole blood drawn from said subject;
incubating the PRP at a temperature greater than room temperature for up to 5 days, wherein said incubating step occurs less than 24 hours after said obtaining step;
detecting the presence of one or more blood-borne microbes in said PRP; and
selecting one or more therapeutic that will specifically inhibit or destroy one or more blood-borne microbe detected in the PRP of said subject.

17. The method of claim 14, wherein said therapeutic is an antibiotic.

18. A method of treating a subject with a microbial infection, comprising the steps of:
obtaining platelet rich plasma (PRP) from a sample of whole blood drawn from said subject;
incubating the PRP at a temperature greater than room temperature for up to 5 days, wherein said incubating step occurs less than 24 hours after said obtaining step;
detecting the presence of one or more blood-borne microbes in said PRP; and
selecting one or more therapeutic that will specifically inhibit or destroy one or more blood-borne microbe detected in the PRP of said subject; and
treating said subject with a therapeutically effective amount of one or more antibiotic specific to one or more blood-borne microbe detected in the PRP of said subject.

19. The method of claim 16, wherein said antibiotic selection results in fewer non-specific antibiotics being used to treat said subject as compared to antibiotic selection based upon blood cultures utilizing whole blood.

20. The method of claim 17, wherein said antibiotic selection reduces the risk of drug-reacted morbidities as compared to antibiotic selection based upon blood cultures utilizing whole blood.

21. The method of claim 1, wherein said detection of the presence of one or more microbes excludes the diagnosis of a viral infection that presents with similar symptoms.

22. The method of claim 19, wherein said viral infection is severe acute respiratory syndrome coronavirus 2 (SARS-CoV-2) infection.
